(19) United States
(12) Patent Application Publication
(10) Pub. No.: US 2011/0172633 A1
(43) Pub. Date: Jul. 14, 2011

(54) THERAPY SYNCHRONIZATION

(75) Inventors: Irfan Z. Ali, Woodbury, MN (US); Ronald J. Petri, Oakdale, MN (US); Scott L. Kalpin, Harris, MN (US)

(73) Assignee: MEDTRONIC, INC., Minneapolis, MN (US)

(21) Appl. No.: 12/986,578

(22) Filed: Jan. 7, 2011

Related U.S. Application Data
(60) Provisional application No. 61/293,284, filed on Jan. 8, 2010.

(51) Int. Cl. A61M 5/168

(52) U.S. Cl. 604/500

(57) ABSTRACT

A system employing multiple implantable therapy delivery devices may be configured to provide coordinated or synchronized therapy by allowing the devices to communicate with each other, directly or indirectly. Fault tolerance or redundancy may be incorporated into the system to allow for correction of failed devices in real time or pseudo real time.

### RELATED APPLICATION

This application claims the benefit of priority to U.S. Provisional Application No. 61/293,284, filed on Jan. 8, 2010, which application is incorporated herein by reference in its entirety.

### FIELD

The present disclosure relates generally to implantable medical devices; in particular to synchronization of therapy between multiple implantable devices.

### BACKGROUND

Implantable medical devices have been employed to treat a variety of diseases. For example, implantable electrical signal generators, such as Medtronic Inc.'s Activa®, Enterra®, InterStim®, Kinetra®, Restore®, and Soletra® lines of implantable neurostimulators, and implantable infusion devices, such as Medtronic Inc.'s SynchroMed® line of infusion devices including the fully programmable SynchroMed® II implantable infusion device, have been used to treat a pain, spasticity, Parkinson's disease, incontinence, and other disorders. Typically, such implantable electrical signal generators and infusion devices have been used individually to treat a disease of a patient.

However, in some cases, it may be desirable to employ more than one device to treat a disease of a patient, or to treat more than one disease in a patient. Proper synchronization of therapy could be important in such cases, particularly if more than one implantable infusion device is implanted in the patient. With more than one infusion device, the likelihood of the patient experiencing an undesired side effect may increase. For example, it may be more likely that the patient experiences a higher than desired cumulative dose of a single therapeutic agent if more than one implanted pump is delivering the same agent. Alternatively, if the multiple infusion devices are delivering different therapeutic agents, it may be desirable to closely coordinate the delivery to avoid undesired interactions between the therapeutic agents. To date, systems and methods for synchronizing therapies between more than one implantable medical device are lacking.

### BRIEF SUMMARY

The present disclosure describes, among other things, methods and systems for coordinating or synchronizing therapy between multiple implantable medical devices. The methods and systems may allow for improved therapeutic efficacy or improved safety.

In various embodiments, a method for synchronizing therapy between a first implantable therapy delivery device and a second implantable therapy delivery device includes providing synchronization operating instructions to at least one of the first and second therapy delivery devices via an external programmer The synchronization operating instructions may be provided to both the first and the second therapy delivery devices, and communication may take place between the first and the second therapy delivery devices to carry out the synchronization operating instructions. In some embodiments, the first therapy delivery device sends to the second therapy delivery device at least a portion of the synchronization operating instructions. Sensors and patient programmers may also play a role in synchronization of therapy delivery.

For example, in numerous embodiments a method for synchronizing therapy between a first implantable therapy delivery device and a second implantable therapy delivery device includes providing synchronization operating instructions to at least one of the first and second therapy delivery devices via an external programmer. The method also includes providing sensed information to at least one of the first and second infusion device and adjusting a dose of therapeutic agent being delivered, or to be delivered, by at least one of the first and second infusion devices based on the sensed information. The dose adjustment is within parameters defined by the synchronization operating instructions. The sensed information may be provided to the first infusion device, and the first infusion device may provide data regarding the sensed information to the second infusion device or may provide information regarding the dose adjustment to the second device. Thus, therapy may be synchronized between the first and second infusion devices while accounting for sensed information.

By way of further example, in some embodiments a method for synchronizing therapy between a first implantable therapy delivery device and a second implantable therapy delivery device includes providing synchronization operating instructions to at least one of the first and second therapy delivery devices via an external programmer. The method also includes providing patient input to at least one of the first and second infusion devices; and adjusting a dose of therapeutic agent being delivered, or to be delivered, by at least one of the first and second infusion devices based on the patient input. The dose adjustment is within parameters defined by the synchronization operating instructions. The patient input may be provided to the first infusion device, and the first infusion device may provide information regarding the patient input to the second infusion device or may provide information regarding the dose adjustment to the second device. Thus, therapy may be synchronized between the first and second infusion devices while accounting for patient input.

In various embodiments, a method includes comparing operating parameters of a first therapy delivery device in a multi-infusion device system with operating parameters of a second therapy delivery device of the system to determine whether the first therapy delivery device has failed. Any suitable method for comparing the operating parameters may be used. For example, the operating parameters of each therapy delivery device in the system may be redundantly stored in all of the therapy delivery devices of the system. The parameters can be in transmitted to each device via an external programmer, such as a clinician programmer, or may be transmitted between therapy delivery devices in the system. If transmitted entirely or in part between devices in the system, the redundant operating parameters can be updated in real time or pseudo real time based on events or changes in the system for a transmitting therapy delivery device; such as, for example, low reservoir conditions, battery conditions, or the like. The method further includes sending operating instructions to the first therapy delivery device from the second device if a determination is made that the first device has failed. The method may also include determining whether the first device is operating according to the instructions sent by the second device. The first device may be inactivated or instructed to operate in safe mode if the first device is not operating according to the instructions sent by the second device. The method may also include adjusting the operating parameters of the second device to compensate for the improper operation of the first device if the first device is not operating according to the instructions sent by the second device.

In numerous embodiments, a method for determining whether an implantable therapy delivery device in a multi-device coordinated therapy system is operating in manner not conforming to the system parameters includes determining what a first, second and third implantable devices of the system consider to be the system parameters. The method further includes determining whether a conflict exists between what the first, second and third implantable devices consider to be the system parameters. If a conflict exists, the system parameters are set as the system parameters of the two non-conflicting devices, provided that there are two non-conflicting devices.

One or more embodiments of synchronized therapy between multiple implantable devices described herein may provide one or more advantages relative to previous systems in which therapy was not synchronized or coordinated. For example, the ability to synchronize delivery of therapy between multiple devices allows for improved therapeutic efficacy that may be specifically tailored to the given patient in real time or pseudo real time. Further, such systems can have built in redundancy or fault tolerance to correct or compensate for device failure in a manner not achievable when therapy is not synchronized. These and other aspects and advantages will be apparent to one of skill in the art from the accompanying detailed description and drawings.

### BRIEF DESCRIPTION OF THE DRAWINGS

FIG. 1 is a diagrammatic representation of a perspective view of an implantable infusion system implanted in a patient.

FIG. 2 is a diagrammatic representation of a perspective view of an implantable electrical signal generator system implanted in a patient.

FIGS. 3-5 are schematic diagrams of communication schemes between a clinician programmer and multiple implantable therapy delivery devices.

FIGS. 6-11 are schematic drawings of communication schemes in systems having multiple implantable therapy delivery devices, a clinician programmer, and sensors.

FIGS. 12-14 are schematic drawings of communication schemes in systems having multiple implantable therapy delivery devices, a clinician programmer, and a patient programmer.

FIGS. 15-18 are schematic drawings of communication schemes in systems having multiple implantable therapy delivery devices, sensors, a clinician programmer, and a patient programmer.

FIGS. 19-22 are flow diagrams of fault tolerance or redundancy processes.

FIG. 23 is a schematic block diagram of a generic device showing some representative components.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope of spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

This disclosure relates to synchronization or coordination of therapy between multiple implantable devices and associated aspects of such synchronization or coordination. In many embodiments, a physician or clinician can provide instructions to one or more implanted device via telemetry, regarding the overall coordinated therapy between the devices. Thus, the parameters within which the devices may operate may be generally constrained by the physician or clinician. However, a degree of flexibility may be allowed within each implanted device and between implanted devices based on these constraints. In various embodiments, the implanted devices communicate with each other to coordinate therapy. However, the devices need not communicate with each other to provide effectively coordinated or synchronized therapy, as the devices may communicate with external devices for such purposes in some embodiments.

In various embodiments, one or more sensors or patient programmers may be involved in the coordination and synchronization of therapy among or between the implanted therapy devices. For example, a patient programmer may allow the patient to provide real time feedback about their condition, which can be used to adjust or modify therapy provided among or between the implanted devices, within the pre-programmed physician or clinician parameters providing ranges or boundaries for overall therapy. Similarly, one or more sensors may provide feedback that can be used to adjust or modify therapy provided among or between the implanted devices.

This disclosure also provides methods and systems for adding fault tolerance or redundancy between various implantable medical devices to allow for continued delivery of coordinated or synchronized therapy between multiple devices when one or more of the devices fail.

In the description that follows, a variety of examples of system configurations that may be used to deliver coordinated or synchronized therapy in accordance with the teachings presented herein are described. First, examples of systems employing multiple implantable infusion devices are described. Then systems employing multiple implantable devices and sensors are described, followed by a discussion of systems employing multiple implantable devices and a patient programmer, and then a discussion of systems employing multiple implantable devices and one or more sensor and a patient programmer is provided. Then a discussion of fault tolerance or redundancy that can be built into the system is provided. It will be understood that the teachings provided below with regard to a system employing multiple implantable therapy delivering devices without sensors or programmers may be applied to systems employing multiple implantable therapy delivering devices and sensors or programmers, and the like.

### System Employing Multiple Implantable Therapy Delivery Devices

Two or more implantable therapy delivery devices may be employed to provide coordinated or synchronized therapy to a patient. As used herein, an “implantable therapy delivery device” is an implantable medical device configured to actively provide a therapeutic effect to a patient. For example, implantable infusion devices that can deliver therapeutic agent to a patient are considered implantable therapy delivery devices. By way of further example, devices capable of delivering therapeutic electrical signals to tissue of a patient, such as neurostimulators, defibrillators, pacemakers, gastro-intestinal stimulators, and the like, are considered implantable therapy delivery devices. Of course, the teachings presented herein may be employed in conjunction with nearly any implantable therapy delivery device.

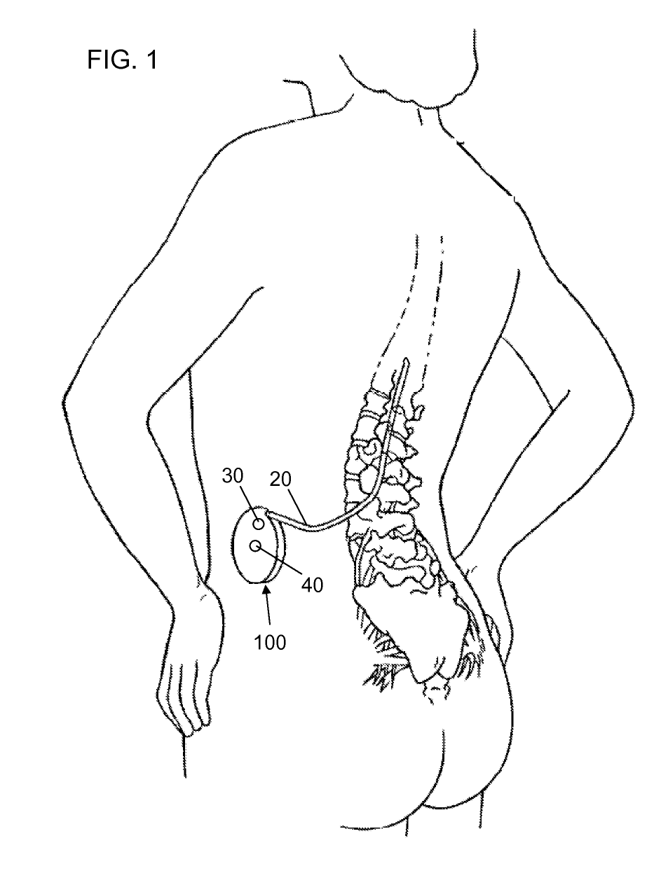

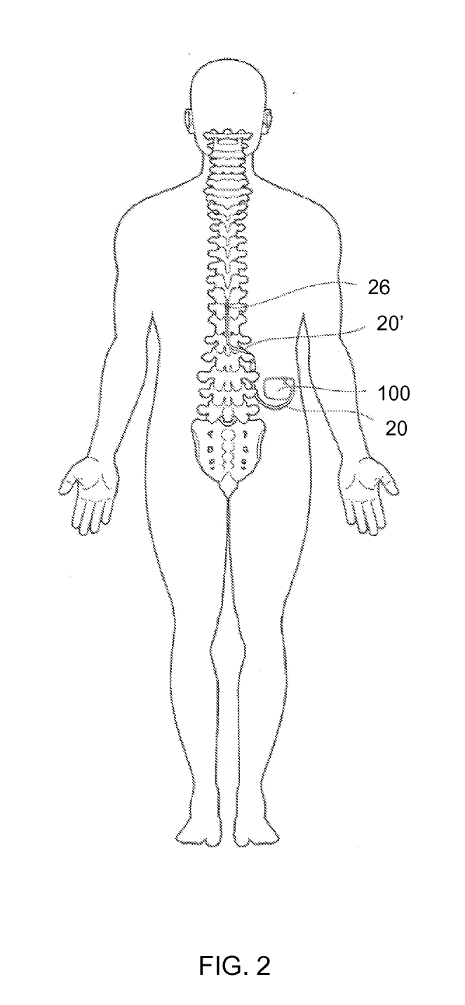

Referring to FIGS. 1 and 2, general representative environments for implanting therapy delivery medical devices 100 and associated devices 20 are shown. Active therapy delivery medical device 100 is depicted as being subcutaneously implanted in an abdominal region of a patient. However, it will be understood that the device 100 may be implanted in any medically acceptable location in the patient, such as behind the patient's ear, in the pectoral region, or in the buttocks, based in part on where the ultimate therapy is to be delivered and patient comfort. A distal portion of associated device 20 is depicted as being intrathecally inserted into the patient's spinal canal through a lumbar puncture and advanced rostrally to a desired location (FIG. 1) or epidurally placed along a suitable location of spinal cord (FIG. 2). Proximal end of associated device 20 is tunneled subcutaneously to location of active device 100, where it may be connected to active device 100. While distal portion of associated device 20 is shown in FIGS. 1 and 2 as being located in or on spinal cord, it will be understood that associate device 20 may be placed at any location in patient for which it is desirable to administer therapy generated or delivered by active medical device 100.

In the embodiment shown in FIG. 1, active implantable device 100 is an infusion device, and associated device 20 is a catheter. Catheter 20 is typically a flexible tube with a lumen running from the proximal end of catheter 20 to one or more delivery regions that are typically located at the distal portion of catheter 20. Proximal portion of catheter 20 is connected to infusion device 20. Distal portion of catheter 20 is positioned at a target location in the patient to deliver fluid containing therapeutic agent from infusion device 100 to patient through catheter 20. Infusion device 100, such as Medtronic Inc.'s SynchroMed™ II implantable programmable pump system, includes a reservoir (not shown) for housing a therapeutic substance and a refill port 40 in fluid communication with reservoir. The reservoir may be refilled by percutaneously inserting a needle (not shown) into patient such that needle enters refill port 40, and fluid containing therapeutic substance may be delivered into reservoir from needle via refill port 40. Infusion device 100 shown in FIG. 1 also includes a catheter access port 30 that is in fluid communication with the catheter 20. Fluid may be injected into or withdrawn from patient through catheter 20 via catheter access port 30 by percutaneously inserting a needle into access port 30. The infusion device 100 may communicate wirelessly, e.g. via telemetry, with a device external to the patient, such as a physician or clinician programmer (not shown in FIG. 1) or other implanted devices (not shown in FIG. 1) as described in more detail below.

In the embodiment shown in FIG. 2, active implantable device 100 is an electrical signal generator, such as Medtronic Inc.'s Restore™ Advanced implantable neurostimulator, and associated devices 20, 20′ are a lead extension 20 and lead 20′. Lead 20′ includes one or more electrical contacts (not shown) on its proximal end portion and one or more electrodes on its distal end portion 26. The contacts and electrodes are electrically coupled via wires running through lead 20′. Electrical signals generated by the signal generator 100 may be delivered to lead 20 through the contacts and then to the patient through the electrodes. As shown in FIG. 2, lead 20′ may be connected to signal generator 100 through a lead extension 20. Extension 20 includes one or more contacts at the proximal and distal end portions that are electrically coupled through wires running through extension 20. Of course it will be understood that with some systems lead 20′ may be directly connected to electrical signal generator 100 without use of a lead extension 20. It will be further understood that more than one lead 20′ or lead extension 20 may be employed per signal generator 100. The signal generator device 100 may communicate wirelessly, e.g. via telemetry, with a device external to the patient, such as a physician or clinician programmer (not shown in FIG. 2) or other implanted devices (not shown in FIG. 2) as described in more detail below.

While FIGS. 1 and 2 depict systems including infusion devices and electrical signal generators as active therapy delivery implantable medical devices 100, it will be understood that the teachings described herein may be applicable to virtually any known or future developed active implantable therapy delivery medical device. While not shown in FIGS. 1-2, it will be understood that the devices 1 will include electronics and software suitable for wireless communication with other devices, whether implanted or external to the patient. Further, it will be understood that the devices 1 will include electronics capable of altering therapeutic output from the device. For an implantable infusion device, the electronics may control the rate of deliver of therapeutic agent. For an implantable signal generator device, the electronics may control relevant therapy parameters such as amplitude, frequency or pulse width of the generated signal.

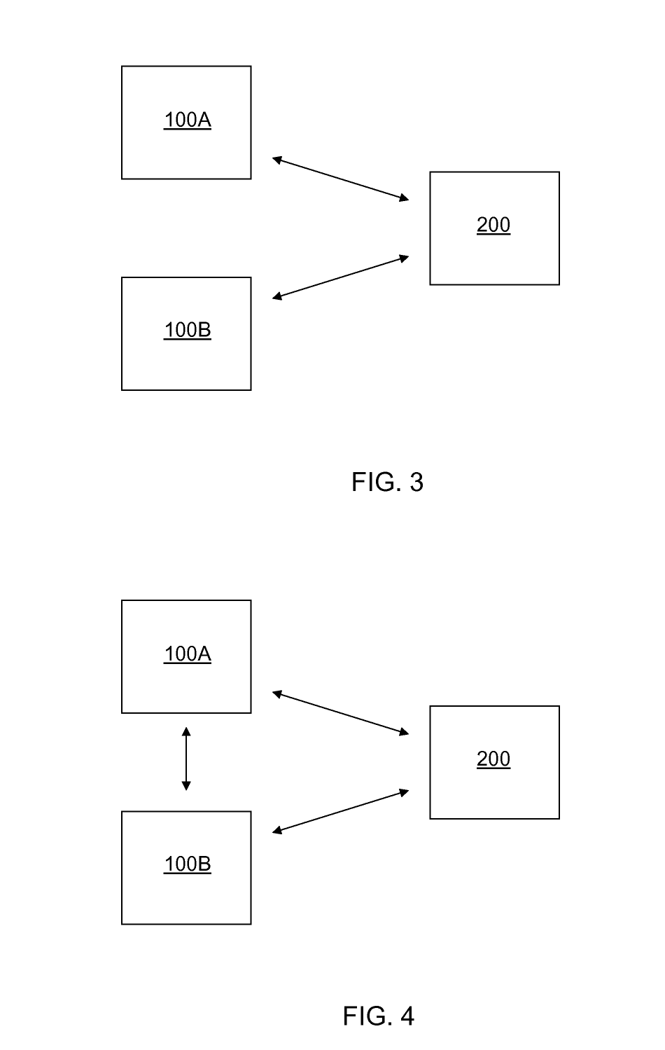

Some examples of configurations that may be employed to coordinate or synchronize therapy between two or more implantable therapy delivery devices are shown in FIGS. 3-5.

Referring now to FIG. 3, multiple implantable therapy delivery devices 100A, 100B are programmed by an external programmer 200 using one or multiple programming sessions whereby the programmer 200 provides programmable therapy delivery parameters to the implantable therapy delivery devices 100A, 100B to perform synchronized drug delivery. The programmer 200 may be any device capable of wirelessly communicating, e.g. via Bluetooth®, or other telemetry standards, with an implanted device 100A, 100B. For example, the programmer 200 may be a Medtronic Inc. Model 7432, Model 9790, N′Vision®, or CareLink® programmer, a personal data assistant device, a laptop or desktop computer, a home monitoring system, or the like.

In the scenario depicted in FIG. 3, the implantable devices 100A, 100B do not communicate with each other, and they deliver therapy based on the parameters provided by the programmer 200. The programmer 200 is the only coordinator for synchronized therapy delivery in this scenario.

Referring now to FIG. 4, multiple implantable therapy delivery devices 100A, 100B are programmed by a programmer 200 using one or multiple programming sessions whereby the programmer 200 provides set of programmable therapy delivery parameters or synchronization algorithm to the implantable devices 100A, 100B to synchronize therapy delivery. The implantable devices 100A, 100B then communicate with each other via telemetry to further synchronize therapy delivery and adjust therapy delivery parameters continuously or periodically after termination of the programming session.

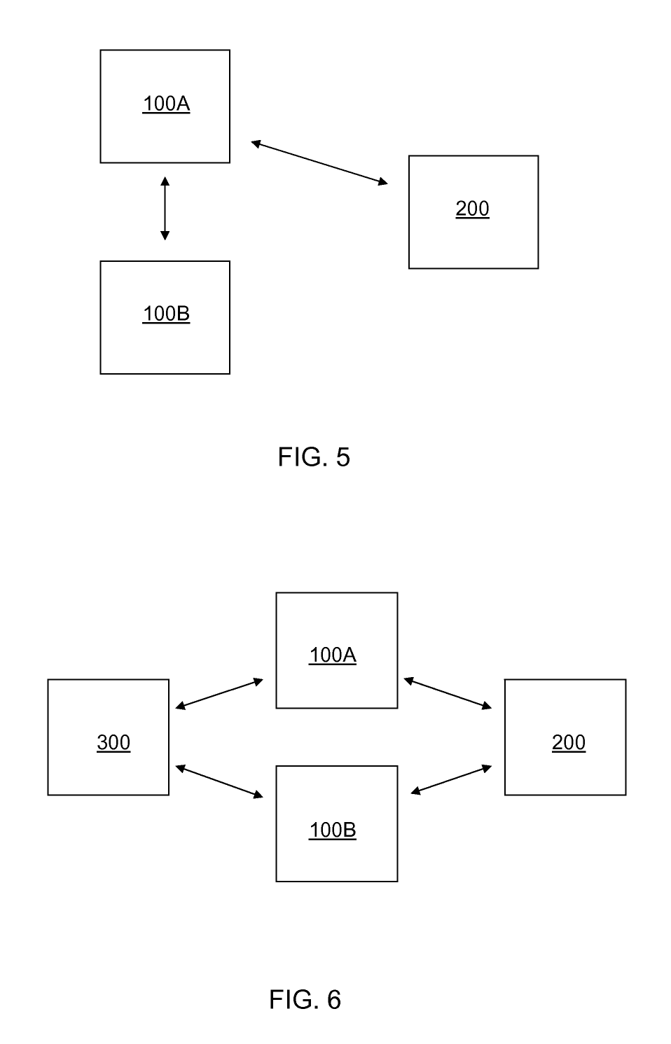

Referring now to FIG. 5, one of the implantable therapy delivery devices 100A in a multiple device configuration is programmed by a programmer 200 using one or multiple programming sessions whereby the programmer provides set of programmable therapy delivery parameters or synchronization algorithm to that device 100A to synchronize therapy delivery. The programmed implantable device 100A then communicates relevant parts of the programming parameters or synchronization algorithm to all the other therapy delivery devices 100B in the configuration via telemetry and the therapy delivery devices 100A, 100B may thus synchronize therapy delivery and adjust therapy delivery parameters continuously or periodically.

In accordance with the scenarios depicted in FIGS. 3-5, the implantable therapy delivery devices 100A, 100B may cooperate in various fashions. For example, the implantable therapy delivery devices 100A, 100B may all be equal peers, and the programmer 200 can communicate with any of the implanted therapy delivery devices 100A, 100B in the configuration with no distinction. Alternatively or in addition, the implanted therapy delivery devices 100A, 100B may be peers that have differences in their functions, and the programmer 200 may choose a given implanted device with which to communicate based on certain rules. Alternatively or in addition, one device (e.g. device 100A in FIG. 5) may be a master device that is responsible for communicating with the programmer 200 and for synchronizing the other implanted devices in the configuration.

It will be understood that the scenarios presented in FIGS. 3-5 are examples, and that any other suitable form of coordination between multiple implantable devices may be employed. For example, there may be multiple master implantable devices that can communicate with a programmer and relay relevant information to slave devices. For example, there may be a master implantable infusion device that provides instructions to one or more slave infusion devices, and there may be a master implantable signal generator that provides instructions to one or more slave infusion devices. Of course, other configurations may be employed.

In some embodiments, therapy is coordinated or synchronized between two or more implantable infusion devices. The infusion devices may deliver the same or different agents or concentrations of an agent to the same general area or to different areas of a patient based, at least partially, on therapeutic considerations. The programmer device may provide instructions to one or more of the infusion devices to provide limits on cumulative dosage, concurrent dosage, dosage rate or the like. Such limits may be based safety or therapeutic considerations.

### System Employing Multiple Implantable Therapy Delivery Devices and One or More Sensors to Coordinate or Synchronize Therapy

One or more sensors may be included in a system for providing coordinated or synchronized therapy between two or more implantable therapy delivery devices. The sensors may be incorporated into the therapy delivery devices or their associated device, e.g. catheters or leads, or may be separate devices, external to the therapy delivery devices, that communicate wirelessly with one or more implantable therapy delivery devices.

Any suitable sensor may be employed. For example, the sensor may be capable of measuring electrical signals generated from a nerve or tissue, may be capable of measuring temperature or impedance of or through a tissue, may be capable of measuring the presence or amount of a chemical species, may be capable of measuring patient activity (e.g. via an accelerometer), may be capable of measuring pressure (e.g., via a pressure transducer), such as pressure in a location of the patient's body or pressure within a catheter of an implantable infusion system, or the like. Typically the sensors employed will be relevant to the therapy provided by the implantable therapy delivery devices. For example, a sensor capable of measuring activity of a nerve may be appropriate when the therapy provided is configured to alter the activity of the nerve. By way of further example, a sensor configured to measure glucose concentration may be appropriate when the therapy is configured to treat diabetes, such as with the delivery of insulin. If the therapy includes delivering a therapeutic agent, such as a drug, a sensor may be configured to detect the amount of the therapeutic agent or a metabolite of the therapeutic agent. In general, any sensor capable of detecting a signal that is relevant to the patient's condition, the therapy, or the effect of the patient on the therapy may be employed.

The sensor may the provide information, directly or indirectly, to one or more of the implantable therapy delivery devices to facilitate coordination and synchronization of therapy in a beneficial manner. Various scenarios in which sensors may be employed in coordinating or synchronizing therapy are shown in FIGS. 6-11.

Referring now to FIG. 6, a system employing an external programmer 200, multiple implantable therapy delivery devices 100A, 100B and a sensor 300 are shown. The configuration shown in FIG. 6 is similar to the configuration shown in FIG. 3 above, with the addition of a sensor 300. In the embodiment depicted in FIG. 6, the therapy delivery devices 100A, 100B do not communicate with each other, but therapy is synchronized via the programmer 200. The sensor 300 may be incorporated into an implantable therapy delivery device 100A, 100B or an associated device such as a catheter or lead, may be a separate implantable device external to the therapy delivery devices 100A, 100B and the associated devices, or may be external to the patient. If the sensor 300 is incorporated into a therapy delivery device 100A, 100B or an associated device, the device into which the sensor 300 is incorporated may use the data retrieved by the sensor 300 to adjust therapy delivered by the device. Sensed information may be communicated to the programmer 200, which may instruct the devices 100A, 100B to modify their coordinated therapy based on the sensed information or programmer 200 may provide pre-programmed parameters and constraints which may be applied by the implantable therapy devices 100A and 100B to the data retrieved from sensor 300 to adjust therapy in real time or pseudo real time. While sensor 300 is shown communicating with both implanted therapy delivery devices 100A, 100B in FIG. 6, it will be understood that sensor 300 need not communicate with both devices 100A, 100B in such a configuration. In embodiments where the sensor 300 is a separate device capable of wirelessly communicating with the implantable therapy delivery devices 100A, 100B it may be desirable for the sensor 300 to communicate with all of the implanted therapy delivery devices 100A, 100B in the system so that each device may adjust therapy delivery based on the sensed information within the constraints provided by the programmer 200. Such a system allows for coordinated and synchronized changes in therapy delivery in real-time or pseudo real time.

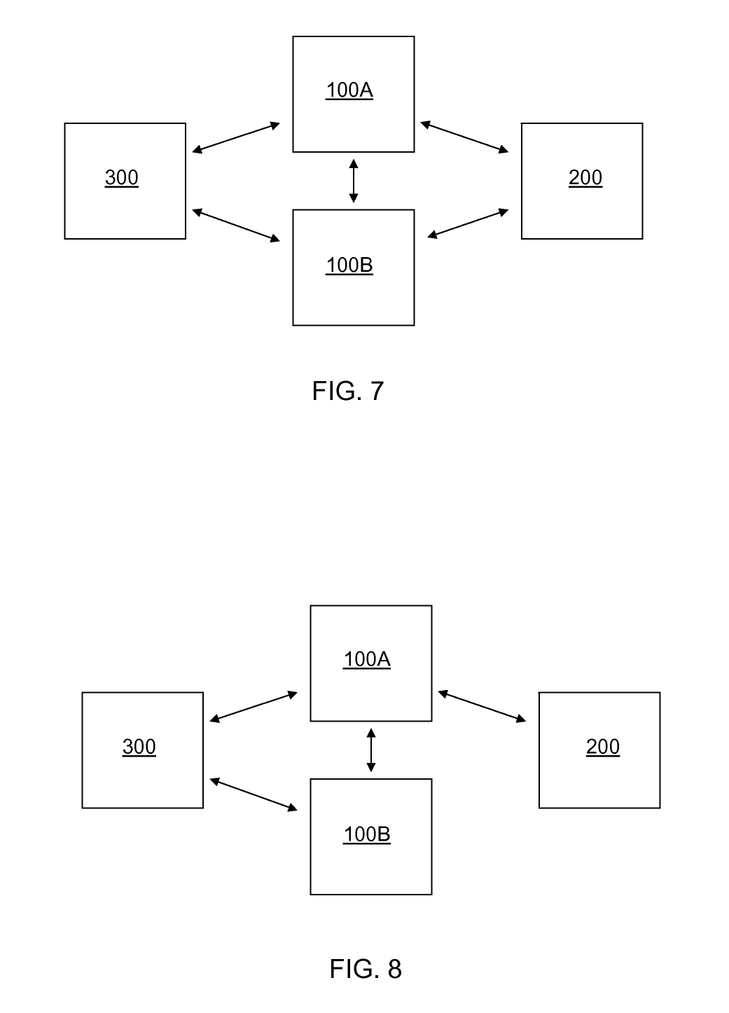

Referring now to FIG. 7, a configuration similar to the configuration of FIG. 4 is shown. In this configuration, the therapy delivery devices 100A, 100B communicate with each other. Accordingly, information obtained from a sensor 300 incorporated into a given therapy delivery device may be communicated in real-time (or pseudo real time) or at a predetermined interval to the other therapy delivery devices so that therapy may be appropriately adjusted among all of the therapy delivery devices 100A, 100B based on the sensed information. Generally, the coordinated adjustments are done within programmed constraints provided by a physician or clinician via programmer 200. As discussed above with regard to FIG. 6, in some embodiments not all of the implanted therapy delivery devices 100A, 100B have incorporated sensors 300. Alternatively or in addition, sensor 300 may be a separate device from implanted therapy delivery devices 100A, 100B and may communicate with one or more of the therapy delivery devices so that therapy may be adjusted as desired.

Referring now to FIG. 8, a configuration similar to the configuration of FIG. 5 is shown. In this configuration, the therapy delivery devices 100A, 100B communicate with each other, with one 100A being a master and the other 100B being a slave. Because the therapy delivery devices 100A, 100B communicate with each other, information obtained from a sensor 300 that is incorporated into one therapy delivery device may be communicated to the other therapy delivery devices so that coordinated adjustments to therapy based on the sensed information may be made. In some embodiments, the slave device 100B may receive information from an integrated sensor 300, may send the master device 100A the sensed information, and may receive instructions from the master 100A regarding adjustments to therapy delivery. In various embodiments, the master device 100A may receive sensed information from an integrated sensor 300 and may send instructions to the slave device 100B regarding therapy adjustments based on the sensed information. In many embodiments, the sensor 300 is a device independent of the therapy delivery devices 100A, 100B and sends information to both therapy delivery devices 100A, 100B (as depicted) or may send information to only the master device 100A.

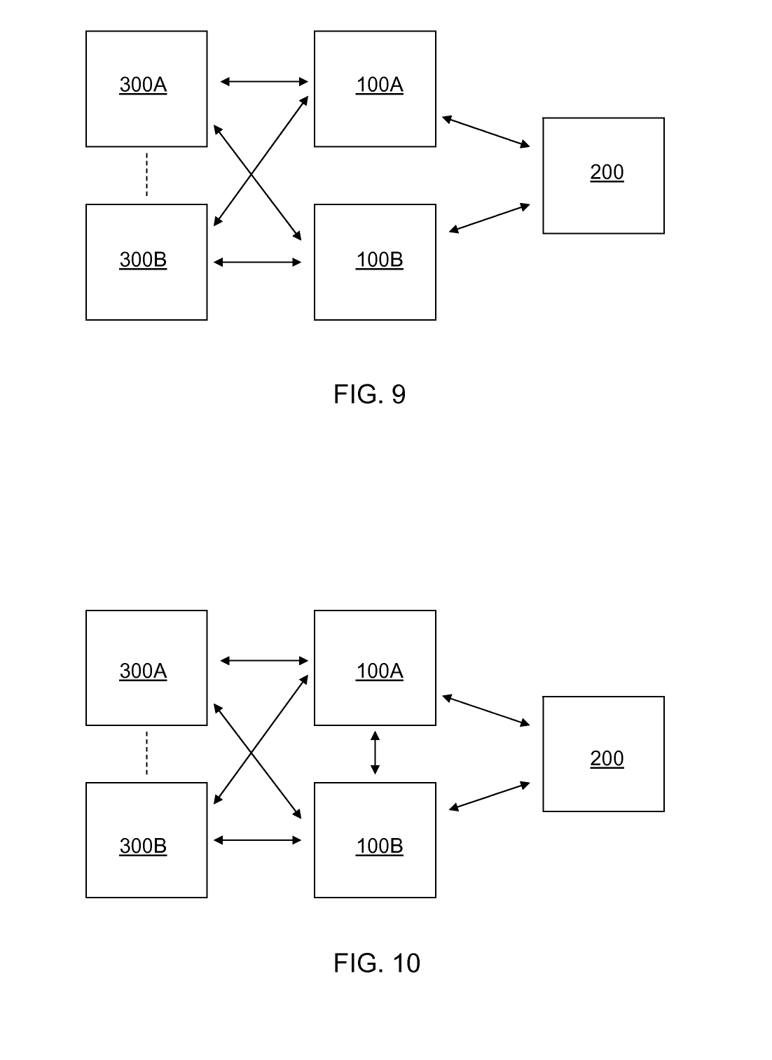

The scenarios depicted in FIGS. 9-11 provide some examples of systems employing more than one sensor 300A, 300B. As indicated above with regard to FIGS. 6-8, the sensors 300A, 300B may be incorporated into the therapy delivery devices 100A, 100B or may be devices separate from the therapy delivery devices 100A, 100B that are capable of communicating with one or more of the therapy delivery devices 100A, 100B. If the sensors are incorporated into therapy delivery devices 100A, 100B or their associated devices, some or all of the therapy delivery devices may include sensors.

In FIG. 9 a scenario similar to that shown in FIG. 3 is shown, in which the therapy delivery devices 100A, 100B do not communicate with each other. In this scenario, information from sensors 300A, 300B (if external) may communicate with all of the therapy devices 100A, 100B so that coordinated adjustments to therapy may be made based on the sensed information, which may be limited by constraints or operating parameters provided by programmer 200. Alternatively or in addition, sensed information may be relayed to programmer 200, which may provide updated therapy delivery instructions to the therapy delivery devices 100A, 100B based on the sensed information. If not all of the therapy delivery devices 100A, 100B receive information from the sensor(s) 300A, 300B, those that do receive sensed information may adjust their therapy delivery parameters based on the sensed information (within the constraints programmed by programmer 200), and the remaining therapy delivery devices 100A, 100B may await further instruction from programmer 200 prior to adjustment of therapeutic output.

In FIG. 10 a scenario similar to that shown in FIG. 4 is shown, in which the therapy delivery devices 100A, 100B communicate with each other. In this scenario, information obtained from a sensor 300A, 300B integrated into a therapy delivery device 100A, 100B may be transmitted to another therapy delivery device 100A, 100B so that therapy adjustments may be coordinated or synchronized within the constraints provided by programmer 200. It will be understood that sensed information, per se, need not be sent, but rather information regarding how one device 100A, 100B is altering therapy or how another device 100A, 100B should alter therapy may be sent. For purposes of this disclosure, each and all of such information is considered “sensed information.” In various embodiments, one or more sensors 300A, 300B are external to therapy delivery devices 100A, 100B and send information to one or more of the therapy delivery devices 100A, 100B so that adjustments to therapy may be made among the devices 100A, 100B based on the sensed information, as desired.

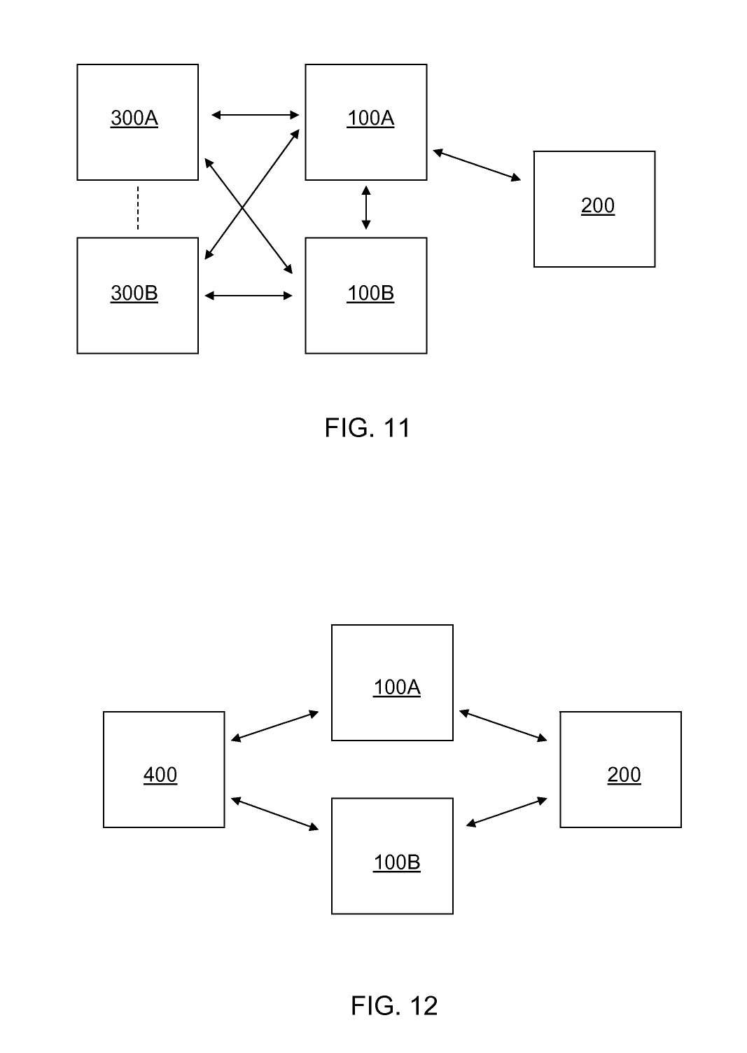

In FIG. 11 a scenario similar to that shown in FIG. 5 is shown, in which the therapy delivery devices 100A, 100B communicate with each other with one device 100A being a master and the other 100B being a slave. In the embodiment depicted in FIG. 11, the master therapy delivery device 100A receives instructions from programmer 200 and provides relevant instructions to the slave therapy delivery device 100B such that the master and slaves coordinate therapy within constraints provided to the master 100A by the programmer 200. Information from sensors 300A, 300B may be communicated in a manner similar to that described above with regard to FIG. 8. Information from sensors 300A, 300B may be transmitted to both the master 100A and the slaves 100B and therapeutic output may be adjusted accordingly within each device or slave 100B may await instructions from master 100A prior to adjusting therapy.

It will be understood that coordinated therapy delivery systems employing sensors and multiple infusion devices configured in manners other than described above and depicted with regard to FIGS. 6-11 are contemplated herein.

### System Employing Multiple Implantable Therapy Delivery Devices and a Patient Programmer to Coordinate or Synchronize Therapy

One or more patient programmers may be included in a system to coordinate or synchronize therapy delivery between two or more implanted therapy delivery devices. Any suitable patient programmer, such as Medtronic Inc.'s MyStim® or MyPTM® patient programmers may be used. Of course any other device capable of wirelessly communicating with and providing instructions to an implanted therapy delivery device may be used. For example, a personal data assistant or a laptop or desktop computer having appropriate software installed may be employed as a patient programmer The use of a patient programmer allows the patient to provide real time feedback about their condition, allowing for coordinated adjustments to therapy to be made as needed or desired. Typically, therapy adjustments made based on patient programmer input are limited by constraints placed on the implanted therapy delivery devices by a physician or clinician via a programmer (e.g., programmer 200 depicted in FIGS. 3-11) to ensure safe and effective therapy is delivered. Further, patient programmers may be used to track patient history and physiological conditions (e.g. pain scores of a patient receiving therapy for treatment of pain) that can be provided to a physician or clinician for evaluation of therapy based on patient experience so that the physician or clinician may make adjustments to the constraints or rules provided to the implanted therapy delivery devices.

In general, a patient programmer may communicate, directly or indirectly (e.g. to slave via master), with multiple implanted therapy delivery devices. The patient may enter information regarding their condition or perceived condition into the patient programmer, which information may then be transmitted to the multiple implanted therapy delivery devices. Based on this information and pre-programmed parameters provided by a clinician during a prior patient visit via a programmer (e.g., programmer 200 depicted in FIGS. 3-11), the multiple therapy delivery devices may provide or update their synchronized therapy delivery.

Examples of system configurations employing a patient programmer are shown in FIGS. 12-14. In the embodiment depicted in FIG. 12, which depicts a scenario similar to FIGS. 3, 6 and 9, multiple implantable therapy delivery devices 100A, 100B communicate with the patient programmer 400 and receive information to modify the therapy, but do not communicate with each other in real time to further synchronize the therapy delivery. However, the implantable therapy delivery devices 100A, 100B use the communication with the patient programmer 400 to synchronize the therapy delivery, in addition to getting information from the programmer 200 to modify the therapy.

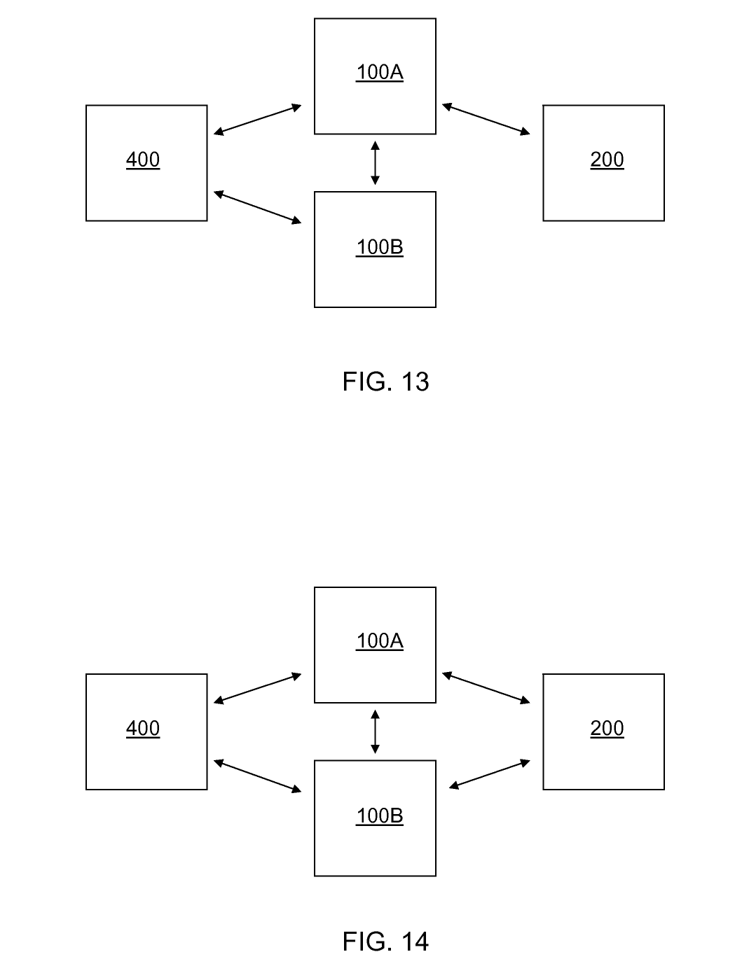

In the embodiment depicted in FIG. 13, which depicts a scenario similar to FIGS. 4, 7 and 10, one therapy delivery device 100A is a master and one or more therapy delivery devices 100B are slaves. The programmer 200 provides overall parameters, rules and constraints to the master 100A, which sends relevant instructions to the slaves 100B. The patient programmer 400 may communicate with both the master 100A and slaves 100B (as depicted) so that synchronized or coordinated changes in therapy delivery may be made. Alternatively, the patient programmer 400 may communicate with the master 100A alone, which can then communicate with the slaves 100B to synchronize updated therapy instructions if needed or desired.

In the embodiment depicted in FIG. 14, which depicts a scenario similar to FIGS. 5, 8 and 11, multiple implantable therapy delivery devices 100A, 100B communicate with each other in addition to communicating with the patient programmer 400 and use the communication between the therapy delivery devices 100A, 100B to further synchronize their therapy delivery in real-time or pseudo real time based on the information provided by the patient programmer 400.

In each of the embodiments depicted in FIGS. 12-14, a clinician via programmer 200 may provide parameters within which the therapy delivery devices 100A, 100B may update the therapy and perform synchronization.

A patient programmer may be used in a system configured to deliver synchronized or coordinated therapy in any suitable manner. For example, in a patient having multiple implanted therapy delivery devices for treatment of pain, the patient programmer may communicate, directly or indirectly, with the implanted therapy delivery devices. The patient may enter a pain score or some other assessment of the patient's condition into the patient programmer, and this information may be transmitted to the implanted therapy delivery devices. The therapy delivery devices may take into account the patient input to alter the therapeutic output of one or more of the therapy delivery devices, provided that the altered therapeutic output is based on and within the constraints provided by the clinician or physician (e.g., pre-programmed parameters and rules transmitted from programmer 200).

For a patient receiving treatment for spasticity via multiple implanted therapy delivery devices, a patient programmer may communicate with one or more of the implanted devices. The patient programmer allows the patient to provide information regarding how spastic they feel (or some other relevant indicator of therapeutic effectiveness or their condition), and this information may be transmitted to the implantable therapy delivery devices, which can take into account this information and adjust therapy in a coordinated fashion, provided that the altered therapeutic output is based on and within the constraints provided by the clinician or physician (e.g., pre-programmed parameters and rules transmitted from programmer 200).

For a patient receiving treatment for Parkinson's disease or another movement disorder via multiple implanted therapy delivery devices, a patient programmer may communicate with one or more of the implanted devices. The patient programmer allows the patient to enter how severe their movement disorder symptoms are (or some other self assessment parameters for their physiological condition), and this information may be transmitted to the therapy delivery devices. The therapy delivery devices may then take into account the patient provided input along with pre-programmed parameters or rules provided by the clinician to alter the therapy and its synchronization

Patient programmer input may also be useful for patients having multiple ailments resulting in multiple devices to manage their conditions. For example, a spastic patient who also suffers from pain may have one implanted device that is more focused on pain management, but which may optionally secondarily provide relief for spasticity, and may have a second device which is primarily focused on spasticity management, but which may optionally secondarily provide relief for pain. The patient can use the patient programmer to provide relevant input, such as their pain score or spasticity score (and/or some other self assessment parameters for their physiological condition). The therapy delivery devices may then take into account the patient provided input along with pre-programmed parameters or rules provided by the clinician to alter the therapy and its synchronization. Depending on whether the patient input indicates pain is worse than spasticity, or vice versa, the therapeutic output of the two implanted device may be modified accordingly.

### System Employing Multiple Implantable Therapy Delivery Devices, a Patient Programmer, and One or More Sensors to Coordinate or Synchronize Therapy

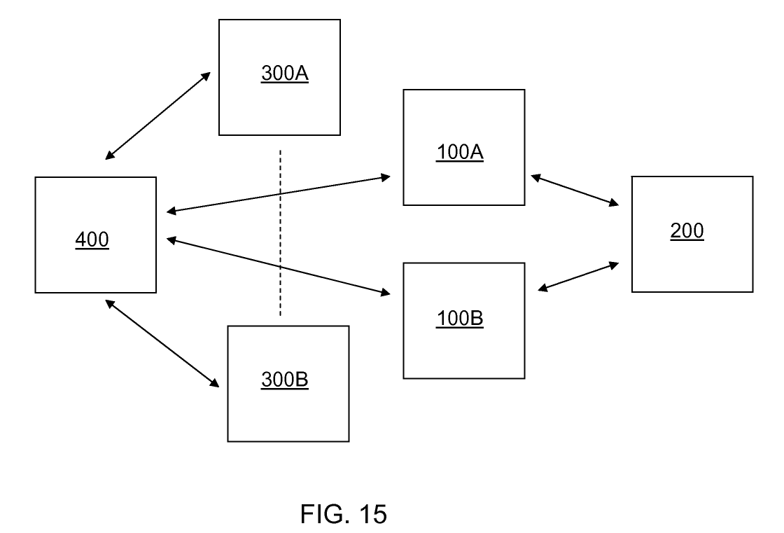

For purposes of illustration various communication schemes or scenarios are described below with regard to FIGS. 15-18 in which a patient programmer 400 and sensors 300A, 300B are employed with multiple implantable therapy delivery devices 100A, 100B to synchronize or coordinate therapy. In systems employing sensors 300A, 300B and patient programmer 400, adjustments in therapeutic output of one or more of the therapy delivery devices 100A, 100B can be made in real time to provide therapy tailored to the patient's needs. As with the various scenarios described above, the ability of the therapy delivery devices 100A, 100B to adjust therapeutic output, individually or collectively, may be limited or constrained by instructions provided by a clinician via programmer 200.

In the embodiments depicted in FIGS. 15-18, a patient has a patient programmer 400 which communicates with multiple implanted devices. The patient could enter information regarding their condition using the patient programmer 400. This information would be transmitted, either directly of indirectly, to the therapy delivery devices 100A, 100B implanted in the patient so that therapeutic output from these devices can be updated or adjusted based on patient input. In addition the patient has sensors 300A, 300B, which may be incorporated into the therapy delivery devices 100A, 100B or their associated devices (e.g. catheter or lead) or may be implanted elsewhere in the patient's body. In some embodiments (which is also the case for the scenarios described above with regard to FIGS. 6-14) a sensor 300A, 300B may have at least a portion that is external to the patient. In some embodiments, a sensor 300A, 300B may be worn by the patient and may transmit sensed information to the implantable therapy delivery devices 100A, 100B, to the clinician programmer 200, to the patient programmer 400, or the like. The sensors 300A, 300B may transmit or stream information on real-time (or pseudo real-time) basis. As patient input is received or sensor data is received, the multiple implanted therapy delivery devices 100A, 100B may update or change their synchronized therapy delivery within the pre-programmed limits set by the clinician or physician.

Referring now to FIG. 15, a scenario in which the implanted therapy delivery devices 100A, 100B do not communicate directly is shown. This scenario is similar to the scenarios depicted in FIGS. 3, 6, 9 and 12. In the scenario depicted in FIG. 15, the implanted therapy delivery devices 100A, 100B may communicate with programmer 200, the sensors 300A, 300B, or the patient programmer 400 to synchronize or update therapeutic output in a coordinated fashion, e.g. as discussed above with regard to the scenarios presented in FIGS. 3, 6, 9 and 12.

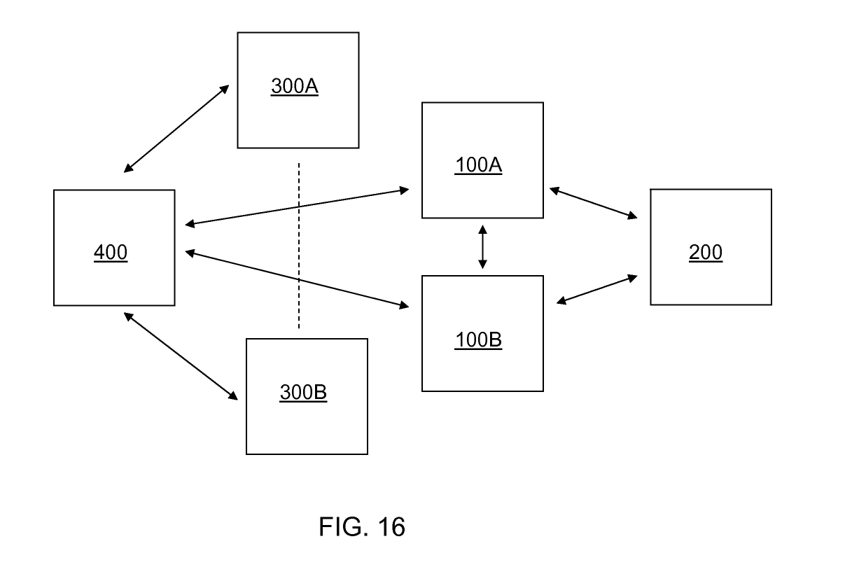

In the embodiments depicted in FIG. 16, the patient programmer 400, in addition to providing patient input, communicates with the sensors 300A, 300B and acts as the main synchronizer of the therapy for the implantable therapy deliver devices 100A, 100B in real time based on input from the multiple sources including the therapy deliver devices 100A, 100B. The clinician programmer 200 may be used to provide general operating guidelines and constraints on the adjustments to therapy that may be coordinated by the patient programmer 400 in this scenario.

In the scenario depicted in FIG. 16, the sensors 300A, 300B may communicate with the patient programmer 400 and stream their data to the patient programmer 400. The patient programmer 400 may then transmit the sensor data to the implanted therapy delivery devices 100A, 100B relaying it as received from the sensor(s) 300A, 300B so that the therapy delivery devices 100A, 100B may determine how to adjust therapy based on the sensed information. Alternatively or in addition, the patient programmer 400 may contain electronics, (e.g., (i) a processor and memory with software, (ii) an application specific integrated circuit (ASIC) and appropriate hardware, or (iii) the like), that are configured to send instructions to one of more of the implanted therapy delivery devices 100A, 100B regarding therapeutic adjustments based on the information received from the sensor(s) 300A, 300B. By using the patient programmer 400 or another external device to process data received from the sensors 300A, 300B and sending the post-processed information to the implanted therapy delivery devices 100A, 100B, the processing burden (including associated power consumption burden) may be desirably taken off of the implanted therapy delivery devices 100A, 100B. Further, it may be more efficient for external devices to initiate communication, relative to implanted devices which may be required to ensure that all channels are clear prior to initiating communication due to regulatory concerns. In addition, signal attenuation within tissue may be significant, so having a patient programmer 400 or other external device receive the information from sensors 300A, 300B or implantable therapy delivery devices 100A, 100B and then broadcast it to other implantable therapy delivery devices 100A, 100B may make the communication more robust and efficient.

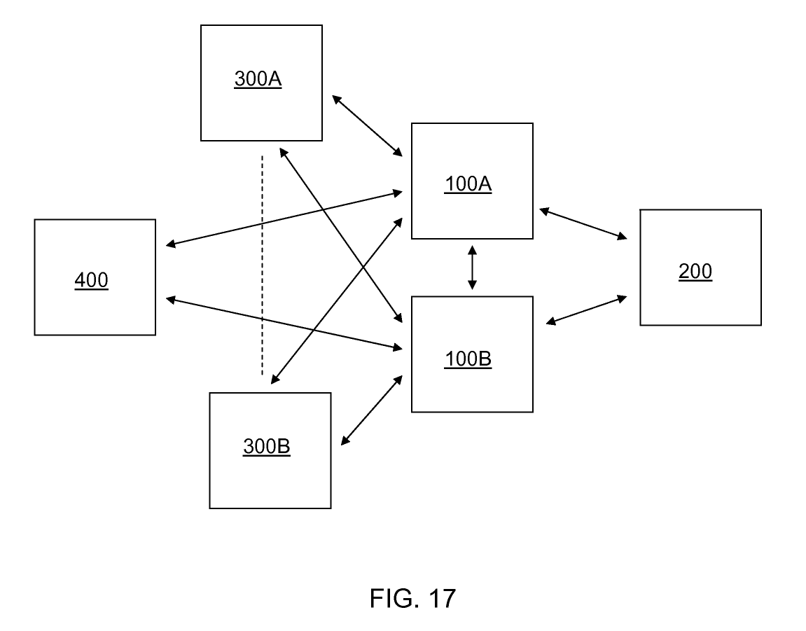

In the scenario depicted in FIG. 17, therapy synchronization or coordination occurs at the level of the implanted therapy delivery devices 100A, 100B based on limitations or instructions provided by a clinician via programmer 200. The multiple therapy delivery devices 100A, 100B communicate with each as well as the patient programmer 400 and the sensor(s) 300A, 300B. The therapy delivery devices 100A, 100B individually or collectively act as the center for all therapy changes and synchronizations among themselves in real-time based on the information provided by the patient programmer 400 and sensors 300A, 300B.

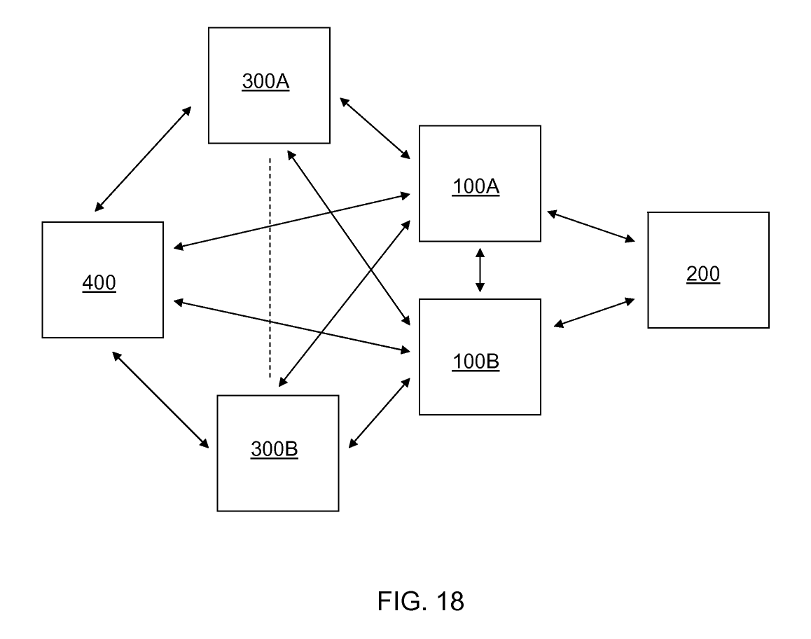

The scenario depicted in FIG. 18 is a hybrid scenario, where only some of the implanted therapy delivery devices 100A, 100B communicate with each other or the patient programmer 400, and some sensors 300A, 300B directly communicate with the therapy delivery devices 100A, 100B and other sensors communicate with the patient programmer 400. The hybrid setup and the determination as to which sensors or therapy delivery devices communicate with each other may be configured by a clinician via programmer 200. In this scenario, each therapy delivery device 100A, 100B may be configured independently, whereby it may optionally communicate with other therapy delivery device, sensors 300A, 300B or patient programmer 400 in any combination. In addition, the patient programmer 400 may also communicate with certain sensors 300A, 300B and perform some processing of the sensor data which is then transmitted to certain therapy delivery devices 100A, 100B.

Similar to systems employing patient programmers as discussed above, systems employing sensors and patient programmers may be used to provide coordinated therapy to treat a variety of diseases in a patient, such as pain, spasticity, Parkinson's disease or multiple ailments in an individual patient. For example, a pain, spasticity, Parkinson's disease or movement disorder patient having multiple implanted therapy delivery devices may use a patient programmer to communicate with the therapy delivery devices or sensors, such as an accelerometer. Information may be streamed to or from the implanted therapy delivery devices or patient programmer. The patient programmer allows the patient to enter pain or spasticity score (or some other relevant self assessment parameters for their physiological condition). This information may be transmitted to the implanted therapy delivery devices. The therapy delivery devices may then take into account the patient-provided input along with the sensor-provided physiological data such as activity, posture, etc and pre-programmed instructions provided by clinicians during a prior programming session to alter the therapy and its synchronization in real time/pseudo real-time.

### Fault Tolerance or Redundancy

In systems employing coordinated therapy among multiple implanted therapy delivery devices, the likelihood that a given device will fail to function properly increases with each device added to the coordinated therapy system. Accordingly, having processes in place to account for such individual device failures may be desirable so that reliable therapy may continue to be delivered to the patient.

With an implantable therapy delivery device there are many situations that can lead to loss of therapy based on device conditions that do not represent a hardware failure but instead are based on other conditions. In some circumstances, it may no longer be safe to operate the device. For example, if the device is an implantable infusion pump it may not be safe to continue to deliver therapeutic agent after a failure has occurred due to potential overdose. If the infusion device has been pre-programmed with an infusion schedule that is time dependent, loss of sense of time may be sufficient to cause the device to shut down or operate under default parameters for purposes of patient safety. However, when multiple therapy delivery devices are employed in a synchronized or coordinated fashion, the ability to restore proper operation of the failed device without the need for a clinician visit and reprogramming by a clinician programmer may be possible and desirable.

As used herein, “failure” of a therapy delivery device means that the device is incapable of operating based on pre-programmed parameters or is unable to verify that it is operating under such parameters. Conditions that may result in device failure include losing information on the device's current state, key therapy or configuration parameters, the device's sense of time or where in time the device should be operating under certain therapeutic parameters, software errors causing memory corruption, environmental conditions such as electromagnetic interference, magnetic resonance imaging, etc. causing memory corruption, or the like.

Some examples of failures that may occur in currently available infusion devices and the ways in which the failures may be overcome in light of the synchronized therapy described herein will now be discussed. For example, following a reset some currently available implantable infusion devices do not continue with flex (time dependent infusion) prescription, but instead either stop or use a fixed infusion rate, because the time base can no longer be trusted. Such measures are configured to provide safety to the patient, but can result in withdrawal symptoms. With coordinated therapy between multiple therapy delivery devices, such as multiple infusion devices, an infusion device in the system that has not experienced a failure can provide the time base for the failed infusion device that underwent reset. In this manner, the failed infusion device can be restored so that it may continue with its regular, pre-programmed infusion schedule.

Similarly, key states for a first implanted infusion device or therapy delivery device may be stored in a second implanted device so that, in case the second device fails, the first device can provide the key states to restore the second device. For example, if an implantable infusion device becomes corrupted such that its life cycle state (e.g., “in manufacturing”, “on the shelf”, “implanted”, etc.) cannot be verified or inappropriately changes, other implanted devices in the system can provide the corrupted device with appropriate information to restore its proper functioning. Such redundancy allows for continued smooth operation of the coordinated system without the patient needing to visit a clinician for reprogramming of the failed device. Other similar therapeutic parameters may also be redundantly stored in case of failure of one or more device.

It will be understood that such redundancy is applicable to systems employing more than two implantable therapy delivery devices. In some embodiments, key states (or other relevant information) for all implanted therapy delivery devices may be stored in all other implanted therapy delivery devices so that in case any one device fails, the other devices can provide the key states to restore the failed device.

In addition, a multiple implantable therapy delivery device system can provide for cross-checking of each other to verify that the devices are operating correctly. If a device is found to not conform to the overall system operating instructions, then the device can be turned off, made to operate in a safe state, restored to conforming operation, or the like. The determination of what constitutes conformation to operating instructions for each device may be based on rules or algorithms including algorithms in case of a tie or deadlock. By way of example, if one therapy delivery device is the master and other are slaves, the master may be used to resolve a conflict as to which device is operating properly. By way of further example, if correctness of key therapeutic parameters, such as infusion rate or signal amplitude are in question, default rules regarding certain ranges or states may be used to determine which device is operating properly and which device has failed. In yet another example, if a majority can be reached (would need three or more devices), the majority may be used as the deciding factor. It will be recognized that other processes for determining whether a device has failed or for correcting a failed device are contemplated herein. It will be further understood that one or more devices may be capable of providing an alarm to the patient or clinician in the event of a device failure. It may be desirable to provide an alarm even if the function of the failed device is restored so that it can be verified that the system has been properly restored.

In various embodiments when the function of a failed device cannot be properly restored (e.g., if there is a hardware failure or software failure that cannot be corrected via the other implantable devices or patient programmer), the remaining devices in the system may alter therapy to compensate for the failed device. For example, if multiple infusion pumps are implanted and configured to deliver the same or different drugs, the remaining properly functioning devices may infuse drug at a higher rate, at least on a short term basis, to compensate for the failed device. The system may be configured to not exceed a certain cumulative dose from the various devices. However, on failure of one infusion device, the others may deliver at a higher rate without exceeding the total cumulative dose due to the loss of the failed device.

Other processes for providing fault tolerance and redundancy based on the various communication schemes described and contemplated herein are also contemplated and would be readily apparent to those skilled in the art based on the disclosure provided herein. It will be understood that the fault tolerance and redundancy processes described herein are merely examples within the intended broader scope of the disclosure.

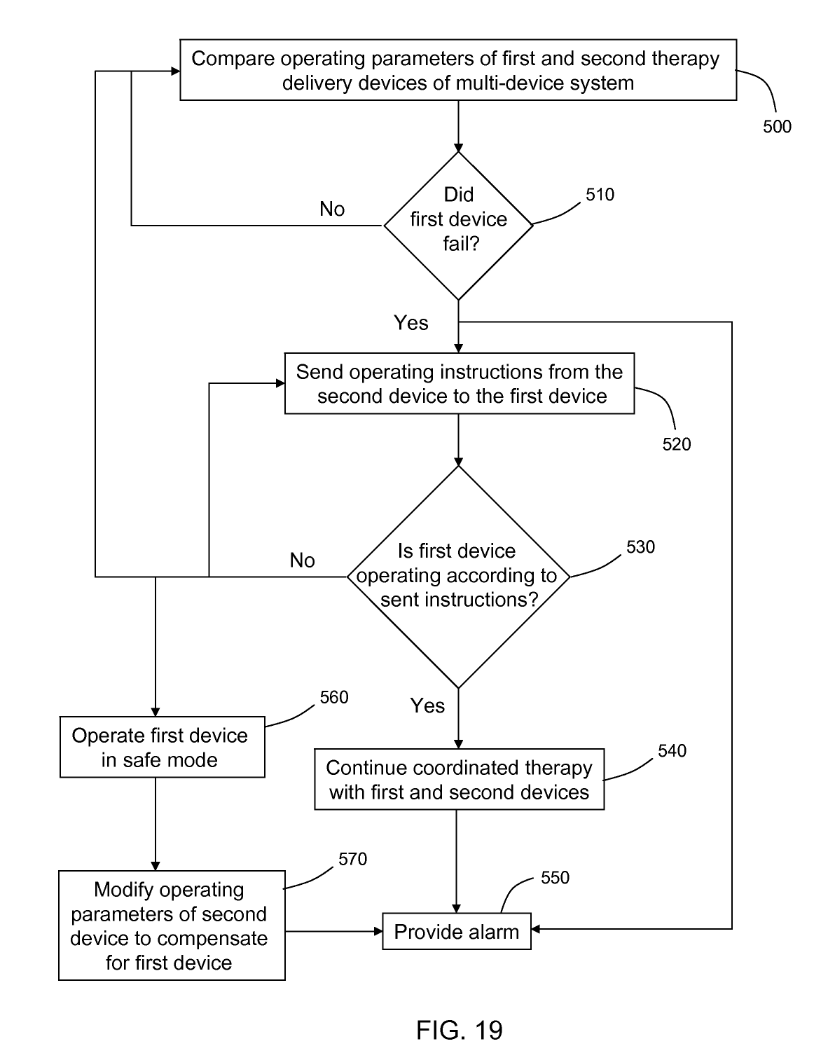

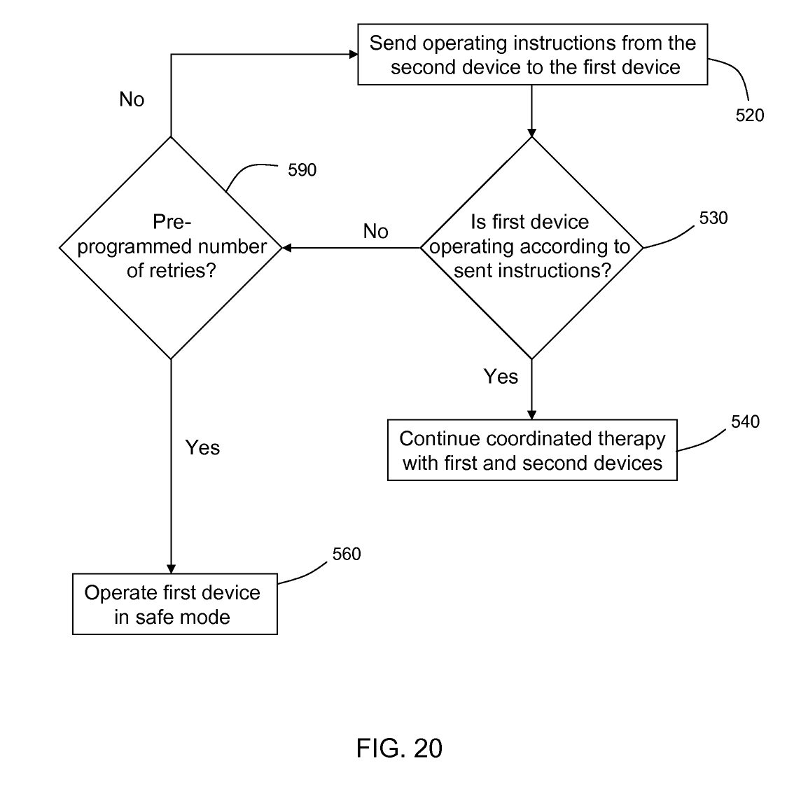

With reference now to FIGS. 19-20, overviews of fault tolerance or redundancy processes are shown. Referring now to FIG. 19, the operating parameters of a first and second device of a multi-therapy delivery device system may be compared (500) to determine whether the first device has failed (510); e.g. is operating outside of system parameters programmed by a clinician programmer, has suffered a memory or system corruption, has lost its sense of time, etc. If the first device is determined to have failed, the second device can send operating instructions (which are broadly referred to herein as any instructions capable of causing the first device to operate in accordance with system instructions) to the first device (520). It may be desirable to determine, at a single time or periodically, whether the first device is operating in accordance with the operating or corrective instructions sent by the second device (530). If the first device continues to operate properly, the coordinated therapy of the first and second device may continue to operate within the constrains of the system parameters (540). However, it may be desirable to provide an alarm (550) to alert the patient or a clinician that a device has failed so that a clinician can verify that the system has been properly corrected.

Still with reference to FIG. 19, if the first device is not operating in accordance with the instructions sent (530), it may be desirable to have the first device operate in safe mode (560), which may include shutting down therapeutic output of the device. It may also be desirable to modify the parameters of the second device to compensate for the failure of the first device (570). As indicated in FIG. 19, if the device is not operating in accordance with the instructions sent (530) the operating instructions may be resent from the second device to the first device (510) or the entire process may be re-initiated. In some embodiments and with reference to FIG. 20, a pre-programmed or predetermined number of retries [e.g., sending instructions (530) and determining whether the first device is operating in accordance with the sent instructions (530)] may be built in to the system. If the pre-programmed or predetermined number of retries (590) has been reached, the first device may be operated in safe mode (560).

Only a few selected steps of FIG. 19 are shown in FIG. 20 for the purposes of convenience. It will be understood that the steps shown in FIG. 20 may be implemented in the method depicted in FIG. 19. It will also be understood that a retry may include the step (500) of comparing operating parameters of the first and second devices depicted in FIG. 19.

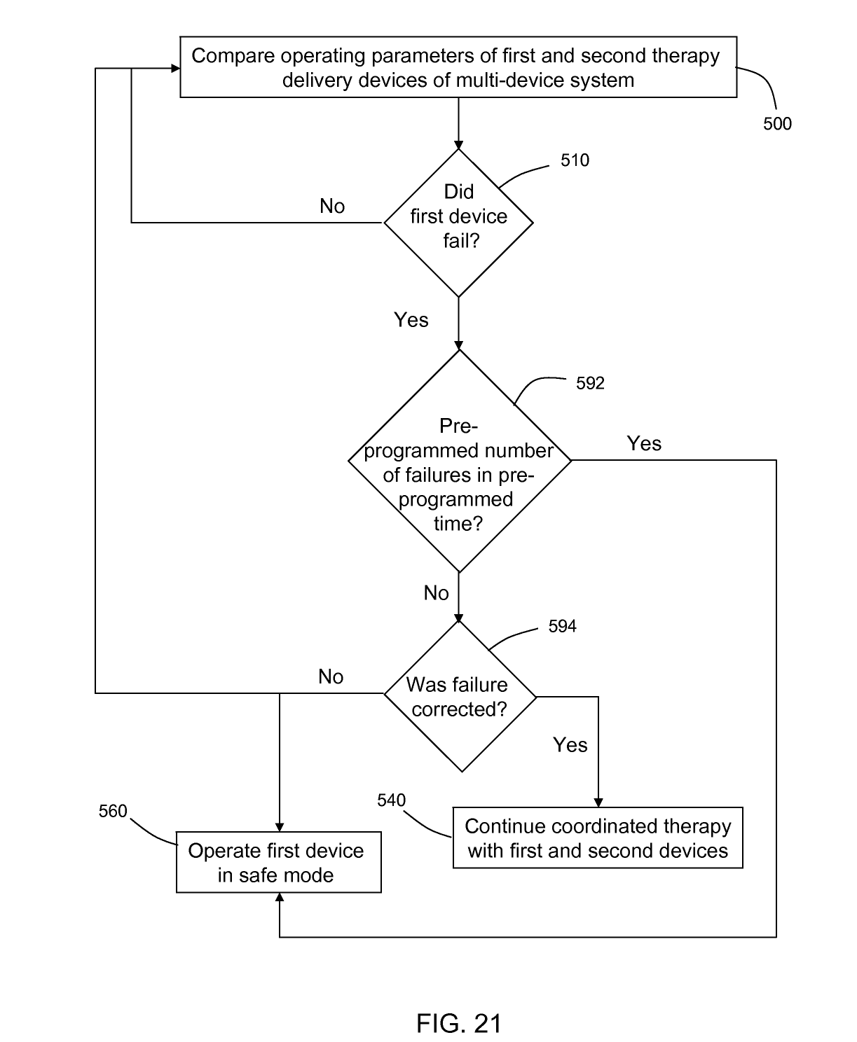

In some situations the first device may appear to have restored its operation or to be operating in accordance with the instructions sent by the second device, but may then revert to a failed state. If this occurs a preprogrammed or predetermined number of times, the first device may be placed in safe mode. For example and with reference to FIG. 21, operating parameters of a first and second device may be compared (500) and a determination may be made as to whether the first device failed (510). If the first device has failed, attempts to correct the failure may be made (e.g. as discussed above with regard to FIGS. 19-20). However, if the first device has failed a preprogrammed or predetermined number of times over a pre-programmed or predetermined duration of time (592), it may be desirable to operate the first device in safe mode (560), even if the failure is correctable. If the first device has not failed a preprogrammed or predetermined number of times over a pre-programmed or predetermined duration of time (592) and if the device failure is corrected (594), the first and second devices may continue to deliver coordinated therapy (540). It will be understood that the predetermined duration of time may be any suitable amount of time, including forever or an infinite amount of time.

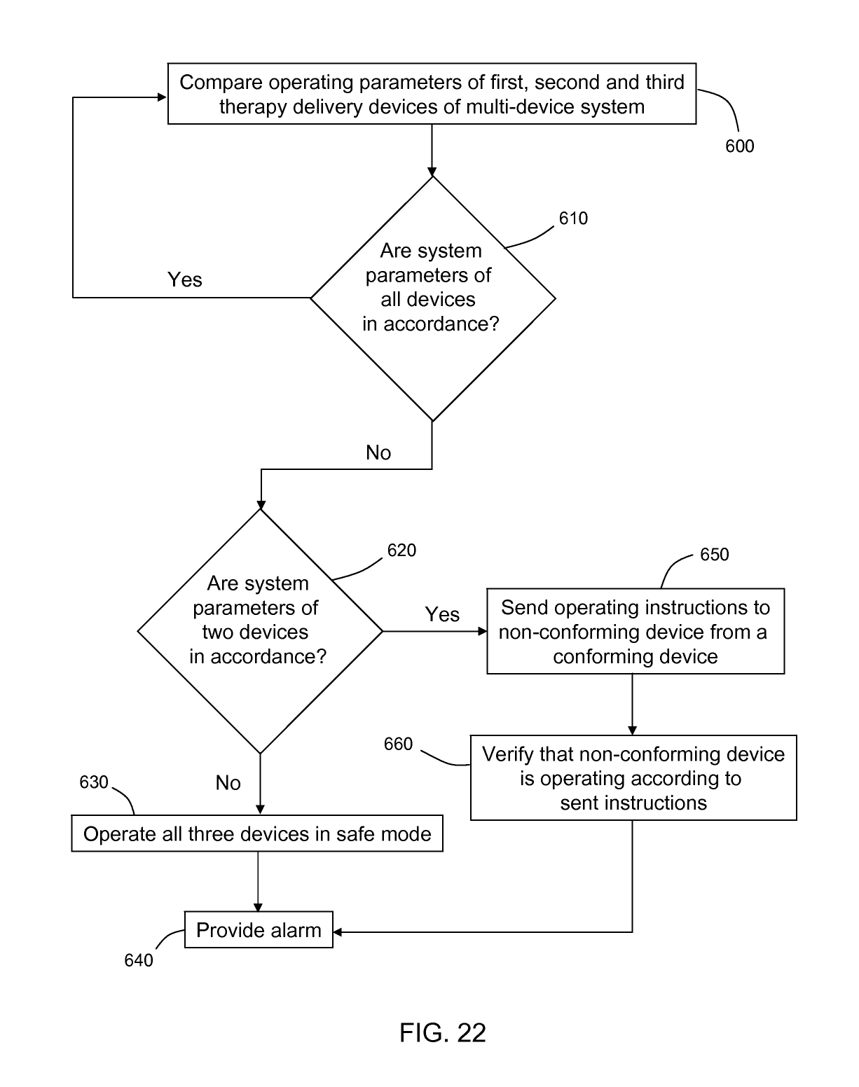

Referring now to FIG. 22, an example of a majority rule process is shown. In the depicted process, the operating parameters of first, second and third therapy delivery devices are compared (600) to determine whether all of the devices are operating in accordance with system instructions (610); e.g. to determine whether there is any conflict in the instructions between the three devices based on their overall system instructions. If all of the devices are not conforming to system parameters, a determination is made as to whether two (a majority) of the three devices are operating in accordance with system parameters (620); e.g., there is no conflict between two of the three devices. If there is a conflict among all of the devices (none are in agreement), then it may be desirable to operate all three devices in safe mode (630). It may also be desirable to provide an alarm (640) so that the patient can be alerted to see a clinician for reprogramming of the devices. In some embodiments, the first, second and third therapy delivery devices have different operating parameters where the operating parameters of each device are redundantly stored on the other devices. The ability to determine whether the devices are operating in accordance with the “system parameters” (610) may then be determined by checking the redundantly stored parameters against the parameters of a given device to determine whether the “system parameters” of the devices are in accordance (620).

Still referring to FIG. 22, if there is agreement between two of the three devices (620), at least one of the confirming devices may send operating instructions to the non-conforming device (650), which may be via a patient programmer or the like, and the proper operation of the non-conforming device may be verified (660); e.g. according to the process outline in FIG. 19 at steps 530-570.

It will be understood that FIGS. 19-22 provide just some examples of the ways in which fault tolerance and redundancy can be built into a system having multiple implantable therapy delivery devices configured to provide synchronized or coordinated therapy. Through communication, direct (e.g. directly between implanted devices) or indirect (e.g., via an external device such as a patient or physician programmer), between devices in such a system, such fault tolerance and redundancy can be realized. In some embodiments, the fault tolerance and redundancy synchronization may take place through implanted devices individually communicating with an external device or through an external device broadcasting synchronization parameters to some or all implanted device. It will be further understood that the discussion with regard to FIGS. 19-22 was limited to two or three devices for purposes of convenience and that the discussion provided here is applicable to systems employing larger numbers of devices. It will also be understood that the labeling of a “first”, “second” and “third” device in connection with FIGS. 19-22 is also for the purpose of convenience and may be arbitrary.

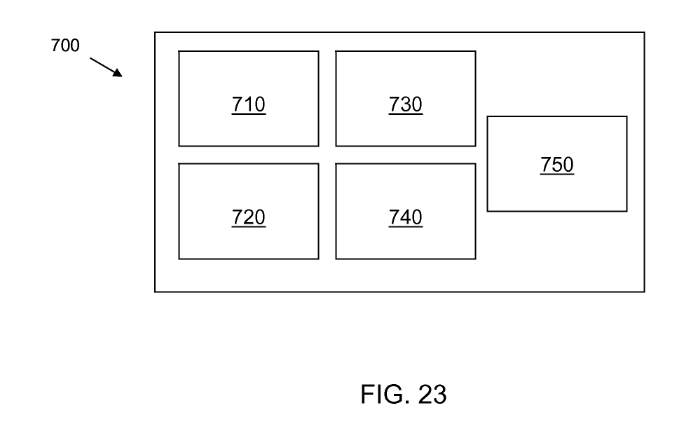

It will be further understood that the various therapy delivery devices, sensors, and programmers may include components that are well known in the art. By way of example and with reference to FIG. 23, a schematic block diagram of a generic device is shown. The device 700 may be a therapy delivery device, sensor or programmer. The device includes a power source 710 such as a primary or rechargeable battery or A/C adaptor (if external). The device 700 may also include a processor 720 and memory 730. The processor 720 may be any variety of processor, such as a microprocessor, microcontroller, a digital signal processor, or application specific integrated circuit (ASIC), depending in part on the function of the device. The memory 730 typically includes volatile memory and nonvolatile memory, such as random access memory, electrically erasable programmable read only memory, or the like. The device 700 also includes a communications module 740 that includes a transmitter or a transmitter and receiver for communicating with other devices. The communications module may be configured to communicate via any wireless technologies, including acoustic or radio frequencies, such as via Bluetooth or Telemetry standards. The device 700 may also include an additional module 750 which may be a sensor module if the device is a sensor device, a therapy module if the device is a therapy delivery device, or the like. The various modules may communicate with each other via a bus, a circuit board traces, or the like.

Also contemplated herein are computer-readable media for carrying out the methods described herein. A computer-readable medium may be non-transitory; e.g., stored in memory as opposed to a fleeting signal. The memory may be in the form of RAM, ROM, or the like. In some embodiments, the computer readable medium is stored on a flask drive, a compact disc, a DVD, a hard-drive, or the like. The computer-readable medium, when executed, causes one or more device in a system described herein to carryout a method described herein, or a part thereof. The devices contain electronics (e.g., processors and memory) that allow the devices to carry out the methods, or parts thereof, when the computer-readable media are executed by the devices.

### Summary of Selected Disclosed Aspects

This disclosure, in various aspects, describes devices, systems, methods and computer readable media.

In a first aspect, a method is described. The method comprises (i) comparing operating parameters of a first infusion device in a multi-infusion device system with operating parameters of a second infusion device of the system to determine whether the first infusion device has failed; and (ii) sending operating instructions to the first infusion device from the second device, if a determination is made that the first device has failed, to attempt to correct the failure of the first device.

A second aspect is a method of the first aspect, further comprising determining whether the first device is operating according to the instructions sent by the second device.

A third aspect is a method of the second aspect, further comprising operating the first device in safe mode if the first device is not operating according to the instructions sent by the second device.

A fourth aspect is a method of the third aspect, wherein the first device is operated in safe mode if a predetermined or preprogrammed number of attempts in a preprogrammed or predetermined duration of time to correct the failure of the first device are made.

A fifth aspect is a method of the third aspect, wherein the first device is operated in safe mode if the first device has been determined to have failed a predetermined or preprogrammed number of times in a preprogrammed or predetermined duration of time.

A sixth aspect is a method of the second aspect, further comprising adjusting the operating parameters of the second device to compensate for the improper operation of the first device if the first device is not operating according to the instructions sent by the second device.

A seventh aspect is a method for determining whether an implantable therapy delivery device in a multi-device coordinated therapy system is operating in manner not conforming to the system parameters. The method comprises (i) determining what a first, second and third implantable devices of the system consider to be the system parameters; (ii) determining whether a conflict exists between what the first, second and third implantable devices consider to be the system parameters; and (iii) setting the system parameters of the first, second and third device as the system parameters of the two non-conflicting devices, if they exist and if a conflict exists.

An eighth aspect is a method of the seventh aspect, further comprising initially providing the system parameters to each of the first, second and third devices.

A ninth aspect is a method of the eighth aspect, wherein the system parameters comprise parameters for operation of each of the first, second and third devices.

A tenth aspect is a method of the ninth aspect, wherein the parameters for operation of each of the first, second and third devices are redundantly stored in each of the first, second and third devices.

An eleventh aspect is a method of the tenth aspect, wherein determining what the first, second and third implantable devices of the system consider to be the system parameters comprises determining what the first device considers to be the operation parameters for the first device, determining what the second device considers to be the operation parameters for the first device, and determining what the third device considers to be the operation parameters for the first device.

A twelfth aspect is a method comprising (i) comparing operating parameters of a first implanted therapy delivery device in a multi-device system with operating parameters of a second implanted therapy delivery device of the system to determine whether the first therapy delivery device has failed; (ii) and sending operating instructions to the first therapy delivery device from the second therapy delivery device if a determination is made that the first device has failed.

A thirteenth aspect is a method of the twelfth aspect, wherein comparing the operating parameters of the first and second device comprises comparing operating parameters of the first device that are redundantly stored on the second device with operating parameters of the first device that are stored on the first device.

A fourteenth aspect is a method for synchronizing therapy between a first implantable infusion device and a second implantable infusion device. The method comprises (i) providing synchronization operating instructions to at least one of the first and second infusion devices via an external programmer; and (ii) communicating between the first and second devices to carry out the synchronization operating instructions.

A fifteenth aspect is a method of the fourteenth aspect, wherein providing the synchronization operating instructions comprises providing the instructions to both the first and the second infusion devices.

A sixteenth aspect is a method of the fourteenth aspect, wherein providing the synchronization operating instructions comprises providing the instructions to the first infusion device, and wherein the communication between the first and second infusion devices comprises the first infusion device sending to the second infusion device at least a portion of the synchronization operating instructions.

A seventeenth aspect is a method of the fourteenth aspect, wherein communicating between the first and second devices comprises communication via an external device.

An eighteenth aspect is a method of the seventeenth aspect, wherein the external device is a patient programmer device.

A nineteenth aspect is a method of the fourteenth aspect, further comprising adjusting a dose of therapeutic agent being delivered, or to be delivered, by one or both of the first and second infusion devices based on the communication between the first and second infusion devices.

A twentieth aspect is a method of the fourteenth aspects, further comprising providing sensed information to at least one of the first and second infusion devices.

A twenty first aspect is a method of the twentieth aspect, wherein the sensed information is provided to the first infusion device, and wherein the first infusion device sends the sensed information to the second infusion device.

A twenty second aspect is a method of the twentieth aspect, wherein a dose of therapeutic agent being delivered, or to be delivered, is adjusted based on the sensed information, wherein the dose adjustment is within parameters defined by the synchronization operating instructions.

A twenty third aspect is a method of the twenty second aspect, wherein the sensed information is provided to the first infusion device, and wherein the first infusion device provides information regarding the dose adjustment to the second device.

A twenty fourth aspect is a method of the twentieth aspect, further comprising providing patient input to at least one of the first and second infusion devices.

A twenty fifth aspect is a method of the twenty fourth aspect, wherein the patient input is provided to the first infusion device, and wherein the first infusion device sends information regarding the patient input to the second infusion device.

A twenty sixth aspect is a method of the twenty fourth aspect, wherein a dose of therapeutic agent being delivered, or to be delivered, is adjusted based on the patient input, wherein the dose adjustment is within parameters defined by the synchronization operating instructions.

A twenty seventh aspect is a method of the twenty sixth aspect, wherein the patient input is provided to the first infusion device, and wherein the first infusion device provides information regarding the dose adjustment to the second device.

A twenty eighth aspect is a method of the fourteenth aspect, further comprising providing patient input to at least one of the first and second infusion devices.

A twenty ninth aspect is a method of the twenty eighth aspect, wherein the patient input is provided to the first infusion device, and wherein the first infusion device sends information regarding the patient input to the second infusion device.

A thirtieth aspect is a method of the twenty eighth aspect, wherein a dose of therapeutic agent being delivered, or to be delivered, is adjusted based on the patient input, wherein the dose adjustment is within parameters defined by the synchronization operating instructions.

A thirty first aspect is a method of the thirtieth aspect, wherein the patient input is provided to the first infusion device, and wherein the first infusion device provides information regarding the dose adjustment to the second device.

A thirty second aspect is a method for synchronizing therapy between a first implantable infusion device and a second implantable infusion device. The method comprises (i) providing synchronization operating instructions to the first and second infusion devices; (ii) providing sensed information to at least one of the first and second infusion devices; and (iii) adjusting a dose of therapeutic agent being delivered, or to be delivered, by at least one of the first and second infusion devices based on the sensed information, wherein the dose adjustment is within parameters defined by the synchronization operating instructions.

A thirty third aspect is a method of the thirty second aspect, wherein providing the synchronization operating instructions comprises providing the instruction to both the first and second infusion device by an external programmer.

A thirty fourth aspect is a method of the thirty second aspect, wherein providing the synchronization operating instructions comprises sending information regarding the synchronization operating instructions from an external programmer to the first infusion device and sending the synchronization operating instructions from the first infusion device to the second infusion device.

A thirty fifth aspect is a method of the thirty second aspect, wherein providing the sensed information comprises sending the sensed information from a sensor to both the first and second infusion devices.

A thirty sixth aspect is a method of the thirty second aspect, wherein providing the sensed information comprises providing the sensed information to the first infusion device, and wherein the first infusion device provides information regarding the dose adjustment to the second device.

A thirty seventh aspect is a method of the thirty second aspect, wherein providing the sensed information comprises providing the sensed information to the first infusion device, and wherein the first infusion device provides the sensed information to the second device.

A thirty eighth aspect is a method of the thirty second aspect, wherein providing the sensed information comprises providing the sensed information to an external device and sending the sensed information to at least one of the first and second infusion devices.

A thirty ninth aspect is a method of the thirty second aspect, wherein sending the sensed information to at least one of the first and second infusion devices comprises sending information regarding the dose adjustment to at least one of the first and second infusion devices.

A fortieth aspect is a method of the thirty second aspect, wherein the sensed information is obtained from a sensor associated with the first device, and wherein the dose of the therapeutic agent being delivered, or to be delivered, is adjusted in the first device.

A forty first aspect is a method of the fortieth aspect, wherein information regarding the adjusted dose in the first device is sent to the second device, and wherein a dose of therapeutic agent being delivered, or to be delivered, from the second device is adjusted based on the synchronization operating instructions.

A forty second aspect is a method of the forty first aspect, wherein the information regarding the adjusted dose in the first device is sent to the second device from the first device.

A forty third aspect is a method of the forty first aspect, wherein the information regarding the adjusted dose in the first device is sent to the second device from an external device.

A forty fourth aspect is a method of the forty first aspect, wherein the dose of therapeutic agent being delivered, or to be delivered, from the second device is adjusted in real time or pseudo real time.

A forty fifth aspect is a method for synchronizing therapy between a first implantable infusion device and a second implantable infusion device. The method comprises (i) providing synchronization operating instructions to at least one of the first and second infusion devices; (ii) providing patient input to at least one of the first and second infusion devices; and (iii) adjusting a dose of therapeutic agent being delivered, or to be delivered, by at least one of the first and second infusion devices based on the patient input, wherein the dose adjustment is within parameters defined by the synchronization operating instructions.

A forty sixth aspect is a method of the forty fifth aspect, wherein providing the synchronization operating instructions comprises providing the instruction to both the first and second infusion device by an external programmer.

A forty seventh aspect is a method of the forty fifth aspect, wherein providing the synchronization operating instructions comprises sending information regarding the synchronization operating instructions from an external programmer to the first infusion device and sending the synchronization operating instructions from the first infusion device to the second infusion device.

A forty eighth aspect is a method of the forty fifth aspect, wherein providing the patient input comprises sending the patient input from a patient programmer to both the first and second infusion devices.

A forty ninth aspect is a method of the forty fifth aspect, wherein providing the patient input comprises providing the patient input to the first infusion device, and wherein the first infusion device provides information regarding the patient input to the second device.

A fiftieth aspect is a method of the forty fifth aspect, wherein the patient input comprises providing the patient input to the first infusion device, and wherein the first infusion device provides the patient input to the second device.

A fifty first aspect is a method of the forty fifth aspect, wherein the patient input is provided to the first infusion device, and wherein the first infusion device provides information regarding the dose adjustment to the second device.

A fifty second aspect is a method for synchronizing therapy between a first implantable therapy delivery device and a second implantable therapy delivery device. The method comprises providing synchronization operating instructions to at least one of the first and second therapy delivery devices via an external programmer.

A fifth third aspect is a method of the fifty second aspect, wherein the first and second devices are implantable infusion devices.

A fifth fourth aspect is a method of the fifty second aspect, wherein the first and second devices are implantable signal generators.

A fifth aspect is a method of the fifty second aspect, wherein the first device is an implantable infusion device and the second device is an implantable signal generator.

Thus, embodiments of THERAPY SYNCHRONIZATION are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method for synchronizing therapy between a first implantable infusion device and a second implantable infusion device, comprising:
providing synchronization operating instructions to at least one of the first and second infusion devices via an external programmer;
providing sensed information to at least one of the first and second infusion devices; and
adjusting a dose of therapeutic agent being delivered, or to be delivered, by at least one of the first and second infusion devices based on the sensed information, wherein the dose adjustment is within parameters defined by the synchronization operating instructions.

2. The method of claim 1, wherein the sensed information is provided to the first infusion device, and wherein the first infusion device provides information regarding the dose adjustment to the second device.

3. The method of claim 1, wherein the sensed information is provided to the first infusion device, and wherein the first infusion device provides data regarding the sensed information to the second infusion device.

4. The method of claim 1, further comprising providing patient input to at least one of the first and second infusion devices.

5. The method of claim 4, wherein the patient input is provided to the first infusion device, and wherein the first infusion device provides the patient input to the second infusion device.

6. The method of claim 4, further comprising adjusting a dose of therapeutic agent being delivered, or to be delivered, by at least one of the first or second infusion devices based on the patient input, wherein the dose adjustment is within parameters defined by the synchronization operating instructions.

7. The method of claim 6, wherein the patient input is provided to the first infusion device, and wherein the first infusion device provides information regarding the dose adjustment to the second device.

8. A method for synchronizing therapy between a first implantable infusion device and a second implantable infusion device, comprising:
providing synchronization operating instructions to at least one of the first and second infusion devices via an external programmer;
providing patient input to at least one of the first and second infusion devices; and
adjusting a dose of therapeutic agent being delivered, or to be delivered, by at least one of the first and second infusion devices based on the patient input, wherein the dose adjustment is within parameters defined by the synchronization operating instructions.

9. The method of claim 8, wherein the patient input is provided to the first infusion device, and wherein the first infusion device provides information regarding the dose adjustment to the second device.

10. The method of claim 8, wherein the patient input is provided to the first infusion device, and wherein the first infusion device sends information regarding the patient input to the second infusion device.

11. A method comprising:
comparing operating parameters of a first infusion device in a multi-infusion device system with operating parameters of a second infusion device of the system to determine whether the first infusion device has failed;
sending operating instructions to the first infusion device from the second device, if a determination is made that the first device has failed, to attempt to correct the failure of the first device.

12. The method of claim 11, further comprising determining whether the first device is operating according to the instructions sent by the second device.

13. The method of claim 12, further comprising operating the first device in safe mode if the first device is not operating according to the instructions sent by the second device.

14. The method of claim 13, wherein the first device is operated in safe mode if a predetermined or preprogrammed number of attempts in a preprogrammed or predetermined duration of time to correct the failure of the first device are made.

15. The method of claim 13, wherein the first device is operated in safe mode if the first device has been determined to have failed a predetermined or preprogrammed number of times in a preprogrammed or predetermined duration of time.

16. The method of claim 12, further comprising adjusting the operating parameters of the second device to compensate for the improper operation of the first device if the first device is not operating according to the instructions sent by the second device.

17. A method for determining whether an implantable therapy delivery device in a multi-device coordinated therapy system is operating in manner not conforming to the system parameters, comprising:
determining what a first, second and third implantable devices of the system consider to be the system parameters; and
determining whether a conflict exists between what the first, second and third implantable devices consider to be the system parameters,
wherein if a conflict exists, the system parameters are set as the system parameters of the two non-conflicting devices if they exist.

18. A method comprising:
comparing operating parameters of a first implanted therapy delivery device in a multi-device system with operating parameters of a second implanted therapy delivery device of the system to determine whether the first therapy delivery device has failed;
sending operating instructions to the first therapy delivery device from the second therapy delivery device if a determination is made that the first device has failed.

19. The method of claim 18, wherein comparing the operating parameters of the first and second device comprises comparing operating parameters of the first device that are redundantly stored on the second device with operating parameters of the first device that are stored on the first device.
